(19) United States
(12) Patent Application Publication
(10) Pub. No.: US 2024/0065547 A1
(43) Pub. Date: Feb. 29, 2024

(54) SYSTEMS AND METHODS FOR ASSESSING EYE HEALTH

(71) Applicant: Lama Al-Aswad, Bronxville, NY (US)

(72) Inventor: Lama Al-Aswad, Bronxville, NY (US)

(21) Appl. No.: 18/238,070

(22) Filed: Aug. 25, 2023

Related U.S. Application Data
(60) Provisional application No. 63/401,209, filed on Aug. 26, 2022.

(51) Int. Cl. A61B 3/14; A61B 3/117; A61B 3/12

(52) U.S. Cl. CPC A61B 3/14; A61B 3/1173; A61B 3/12

(57) ABSTRACT

The disclosure relates generally to the fields of optometry and ophthalmology, and, more particularly, to a system for providing a remote ophthalmologic examination and assessment of a patient's eyes.

### CROSS-REFERENCE TO RELATED APPLICATIONS

This application claims priority to, and the benefit of, U.S. Provisional Application No. 63/401,209, filed Aug. 26, 2022, the content of which is incorporated by reference herein in its entirety.

### TECHNICAL FIELD

The disclosure relates generally to the fields of optometry and ophthalmology, and, more particularly, to a system for providing a remote ophthalmologic examination and assessment of a patient's eyes.

### BACKGROUND

In today's age of increased health awareness, it is important for individuals to routinely make visits to medical practitioners for a range of tests and check-ups, which can provide early detection and prevention of various medical conditions, disorders, and diseases. For example, it is generally recommended that individuals make regular visits (e.g., once every year, two years, etc.) to practitioners in connection with the monitoring, diagnosis, and treatment of medical conditions in number of areas, such as checking heart-health, detecting various cancers, and monitoring for certain genetically predisposed disorders.

One area in which there is a particular need for regular visits to a medical professional is in connection with the examination of an individual's eyes. Typically, the examination of a person's eyes involves the performance of one or more tests for monitoring and diagnosing eye health, such as detecting glaucoma and retinal disorders, inspecting the pupil, and measuring corneal sensitivity, and/or tests for evaluating visual ability and acuity, such as determining refractive error and detecting color blindness.

There are a number of important benefits to obtaining eye health examinations and/or vision examinations on a regular and continual basis. For example, as with many other types of medical examinations, regular visits and checkups by an individual enable practitioners to monitor and track the health of the individual's eyes and to detect and diagnose certain disorders, diseases and other changes in the patient's eyes and/or vision. Significantly, this allows for early detection, diagnosis and treatment of many conditions, which, in turn, frequently increases the likelihood that the treatment will be successful. In fact, many disorders and diseases are generally treatable or even preventable when detected and diagnosed in the early stages. Also, it is well known that changes in vision can often occur somewhat suddenly, such as at certain periods in a person's life, and eyesight can deteriorate continually over time. Accordingly, another important benefit to regular eye examinations is that they help to ensure that optical prescriptions for individuals are up to date and as accurate as possible.

Given the numerous benefits associated with regular eye examinations, it is not surprising that it is typically recommended that individuals visit eye care professionals once every one or two years. Moreover, the need to receive regular eye examinations is particularly important for certain individuals, including those who have a higher likelihood of suffering from various disorders and diseases based on their demographics or other characteristics, such as age, race, profession, individual and/or family history of diseases or disorders, etc. As a result, it is often recommended that many such individuals receive eye exams at least once a year or on an even more frequent basis.

Despite the known importance of regular eye health checkups and vision examinations, many individuals only visit eye care professionals and receive eye examinations on a highly sporadic basis. Other individuals fail to visit an eye care professional at all, or only do so in response to suffering from a medical condition or recognizing a potential problem with their vision.

While there are various contributing factors, primary reasons why many individuals fail to regularly undergo eye examinations involve time involved, cost, and convenience. Typically, in order to obtain an eye examination, an individual is required to expend time and effort to seek out, select, and make an appointment with an appropriate eye care professional. In turn, time is spent traveling to and from the practitioner's office, waiting for the practitioner, partaking in discussions with the practitioner and/or nurse or assistant and undergoing the examination. Similarly, from the practitioner's perspective, the number of patients that can be seen and examined are limited by a number of factors, such as the time required to examine each patient, update the patient's records, and prepare equipment. As a result, individuals are frequently forced to visit eye care professionals at inconvenient times and/or travel to other, less conveniently located professionals. Also, in order to maximize the number of patients that can be seen, practitioners may limit the number of tests and procedures and/or the time spent on such tests, thereby reducing the time needed for each patient. Thus, making regular visits to an eye care professional can often be a time-consuming, inconvenient and expensive commitment.

There have been some attempts in the past to provide systems that simplify and automate the vision testing and examination process. Such systems, however, have significant drawbacks and limitations, which have resulted in their failure to be adopted by consumers in any meaningful way. One such drawback is that many of these systems only provide vision screening or visual acuity testing. Similarly, many of these systems are limited to a restricted or incomplete set of procedures and tests and do not allow individuals to obtain a comprehensive eye examination. Another drawback is that many of these systems require an on-site eye care practitioner and/or operator to provide some or all of the examination.

For example, due to the optical complexity of the eye, current devices (i.e., cameras and the like) used in clinical settings are both large and difficult to use, requiring patient cooperation and trained imagers to operate such cameras. Such cameras are neither remote nor automated. Furthermore, such devices, which commonly include slit-lamps and fundus cameras, require multiple mirrors, light sources, and lenses, and are positioned at focal lengths that can be a foot away from the patient, necessitating a sizeable footprint.

Furthermore, current portable devices for capturing eye images are expensive, sacrifice quality for size, and are unable to image both the anterior and posterior of the eye, which is crucial for providing a comprehensive ophthalmologic examination and assessment of eye health. Furthermore, in-clinic imaging, while of higher quality, requires the use of multiple bulky, expensive tools and also require trained operators.

Developing a wearable compact device that images the whole eye poses significant technical challenges. Some challenges include the ergonomics, type of optics (lenses and prisms) and configurations, the type of bulbs and lighting and configurations, weight, heat output, and further accommodating the overall anatomy of the eye, including the various angles and controlling the pupil size so as to be able to image the fundus of the eye. Currently, there are no solutions for imaging both the anterior and posterior of an eye in a single device. Portable devices for either anterior or posterior imaging, do so at the expense of image quality. Furthermore, like in-clinic devices, such devices tend to be costly and require a trained imager/provider. Such pitfalls make the current offerings unsuitable for remote, home use.

Therefore, there is a need to develop a wearable compact device that is able to capture images of the whole eye so as to provide sufficient image data for a comprehensive assessment of eye health.

### SUMMARY

The present invention recognizes the drawbacks of current eye testing and evaluation systems. To address such drawbacks, the present invention provides a system including a portable, wearable headset allowing a person to perform self-administered collection of eye image data for use in a remote ophthalmologic examination and assessment of a person's eye health.

In particular, the wearable headset of the present invention is a small, portable, and low-cost eye-imaging device that combines the functions of multiple imaging devices, without sacrificing quality. The headset is capable of capturing images of both the anterior and posterior segments of a person's eye without requiring involvement of a trained operator or technician. This portability and self-imaging capability allows for a person to capture digital images of their eyes without having to travel to a clinical setting and obtain assistance. Rather, a person can capture images from the comfort of their home in a relatively automated fashion. The invention further allows for the digital images to be provided to a computing system operably associated with the headset and which provides an interactive platform with which a medical professional is able to interact and analyze the one or more digital images for diagnosis and monitoring of a condition status of the at least one of the patient's eyes.

Accordingly, the system of the present invention, including the wearable headset, enables patients to undergo a complete and fully automated eye examination, while in a fully remote manner. The portability and self-imaging capacity of the wearable headset will allow providers to conduct home-based examinations, thereby removing barriers associated with the current paradigm of in-clinic eye care. This technology allows providers to screen and monitor for potentially blinding conditions such as age-related macular degeneration, diabetic retinopathy, and glaucoma, all in a remote manner. In some embodiments, the system may further be configured to provide automated analysis of the digital images and diagnose a condition status of an eye, including early diagnosis and treatment of an eye disease, based on artificial intelligence techniques.

One aspect of the present invention includes a portable, wearable headset for use in providing a remote and self-administered collection of data for use in an ophthalmologic examination and assessment of one or more eyes of a person wearing the headset. The headset includes a first optical imaging assembly for capturing one or more images of an anterior segment of at least one of the person's eyes and a second optical imaging assembly for capturing one or more images of a posterior segment of at least one of the person's eyes.

The first optical imaging assembly is configured to capture image data providing visualization of one or more structures within the anterior segment of an eye. The one or more structures within the anterior segment comprise at least one of a cornea, iris, ciliary body, and lens. The second optical imaging assembly is configured to capture image data providing visualization of one or more structures within the posterior segment of an eye. The one or more structures within the posterior segment comprise at least one of vitreous humor, retina, choroid, and optic nerve.

The headset comprises a frame supporting the first and second optical imaging assemblies relative to the person's eyes. The specific ergonomics of the headset allow for the headset to be inverted relative to the patient's eyes to allow for capturing images of the anterior and posterior segments. For example, when in a first orientation, the first and second optical imaging assemblies are positioned relative to the person's right and left eyes, respectively. When in a second orientation, the first and second optical imaging assemblies are positioned relative to the left and right eyes, respectively. The frame comprises an invertible nose bridge provided between the first and second optical imaging assemblies. As such, the headset can be worn in the first and second orientations by rotating the headset 180 degrees in a plane of the first and second optical imaging assemblies.

More specifically, the invertible nose bridge comprises a first recess and an opposing second recess, each of the first and second recesses being shaped and/or sized to receive a portion of the person's nose and are symmetrical relative to one another. Accordingly, when in the first orientation, the first recess is positioned adjacent to an upper portion of the person's nose and the second recess is positioned adjacent to a lower portion of the person's nose, and when in the second orientation, the second recess is positioned adjacent to the upper portion of the person's nose and the first recess is positioned adjacent to the lower portion of the person's nose.

Accordingly, when in the first orientation, the first optical imaging assembly is configured to capture image data providing visualization of one or more structures within the anterior segment of the right eye and the second optical imaging assembly is configured to capture image data providing visualization of one or more structures within the posterior segment of the left eye. When in the second orientation, the first optical imaging assembly is configured to capture image data providing visualization of one or more structures within the posterior segment of the left eye and the second optical imaging assembly is configured to capture image data providing visualization of one or more structures within the anterior segment of the right eye.

The first optical imaging assembly of the headset comprises a slit lamp module. The slit lamp module comprises at least a 90-degree slit lamp assembly and a 45-degree slit lamp assembly. The slit lamp module further comprises an imaging assembly for capturing one or more images of the anterior segment of a respective eye illuminated via the 90-degree and 45-degree slit lamp assemblies.

The second optical imaging assembly comprises a fundus camera module. The fundus camera module comprises a fundus illumination assembly including a light source and at least one optical element for projecting illumination upon the posterior segment of a respective eye. The fundus camera module further comprises an imaging assembly for capturing one or more images of the posterior segment illuminated via the fundus illumination assembly.

The wearable headset may further include a communication module for permitting the exchange of data between a computing device and the first and second optical imaging assemblies. The communication module is configured to permit wired and/or wireless transmission of data between the computing device and the first and second optical imaging assemblies. The computing device may include, for example, a remote server configured to receive the one or more images captured via the first and second optical imaging assemblies for use in an ophthalmologic examination of the person's eyes.

Another aspect of the present invention includes a system for providing remote ophthalmologic examination and assessment of a patient's eyes based on the one or more images captured via the wearable headset. The system is configured to collect and process data associated with the digital images captured via the first and second optical imaging assemblies and provide subsequent eye health assessments. For example, the system may include a computing system configured to communicate with the remote wearable headset and receive, from the remote wearable headset, one or more digital images of anterior and posterior segments of at least one of the patient's eyes and provide an interactive platform with which a medical professional is able to interact with and analyze the one or more digital images for diagnosis and monitoring of a condition status of the at least one of the patient's eyes.

The condition status of a patient's eyes may be noted as a normal condition or an abnormal condition. For example, an abnormal condition may include, or is otherwise associated with, a disease. The disease may be associated with the eye, such as age-related macular degeneration or glaucoma. In some instances, the disease may include diabetes mellitus. In particular, the condition may include diabetic retinopathy.

The one or more digital images received from the wearable headset provide visualization of one or more structures within the anterior segment of an eye and one or more structures within the posterior segment of an eye. For example, the one or more structures within the anterior segment may include, but are not limited to, a cornea, iris, ciliary body, and lens. The one or more structures within the posterior segment many include, but are not limited to, vitreous humor, retina, choroid, and optic nerve.

The computing system may grant a medical professional access to the one or more digital images based, at least in part, on HIPAA-compliant security measures. The interactive platform may generally provide for scheduling of remote, virtual meetings between the patient and medical professional. In some instances, the remote, virtual meeting may be synchronized with real time capturing of the one or more digital images via the remote, wearable headset. For example, the computing system may be configured to receive the one or more digital images from the wearable headset in real, or near-real, time during the remote, virtual meeting and the medical professional is able to interact with the one or more digital images via the interactive platform during the remote, virtual meeting.

The computing system may further be configured to output a report providing a diagnosis of a condition status of the at least one of the patient's eyes. The report may further include a suggested course of treatment.

In some aspects, the computing system may be configured to provide automated or semi-automated analysis of the one or more digital images and diagnosis of a condition status based on the analysis by utilizing artificial learning techniques.

For example, the automated or semi-automated analysis may include correlating image data associated with the one or more digital images and reference ocular image data. In particular, the computing system may be configured to run a neural network that has been trained using a plurality of training data sets, each training data set comprises reference ocular image data associated with known eye structures and known conditions associated with the known eye structures. The computing system may be configured to identify, based on the analysis, one or more eye structures in the one or more digital images and an associated condition of the one or more eye structures based, at least in part, on the correlation of image data of the one or more digital images and reference ocular image data.

In one embodiment, the computing system may include a machine learning system selected from the group consisting of a neural network, a random forest, a support vector machine, a Bayesian classifier, a Hidden Markov model, an independent component analysis method, and a clustering method.

In some embodiments, the computing system may include an autonomous machine learning system that associates the known conditions with the reference ocular image data. For example, the machine learning system may include a deep learning neural network that includes an input layer, a plurality of hidden layers, and an output layer. The autonomous machine learning system may represent the training data set using a plurality of features, wherein each feature comprises a feature vector. The autonomous machine learning system may include a convolutional neural network (CNN), for example.

### BRIEF DESCRIPTION OF THE DRAWINGS

FIGS. 1A and 1B are diagrammatic illustrations of a system for providing a remote ophthalmologic examination and assessment of a patient's eyes, including a portable, wearable headset allowing a person to perform self-administered collection of eye image data and an eye assessment system operably associated with the headset and which allows for analysis and subsequent assessment of a health of the patient's eyes.

FIG. 2 is a block diagram illustrating a system for providing a remote ophthalmologic examination and assessment of a patient's eyes consistent with the present disclosure.

FIGS. 3A, 3B, and 3C show front facing, side, and plan views of a wearable headset consistent with the present disclosure fitted upon a person's face and positioning first and second optical imaging assemblies over the respective eyes.

FIGS. 4A, 4B, and 4C are perspective, front facing, and plan views of the wearable headset illustrating the various components of the first and second optical imaging assemblies.

FIG. 5 is a perspective view of the wearable headset illustrating the invertible nose bridge of the frame of the headset which allows for the headset to be inverted such that, upon rotating the headset 180 degrees, the first and second optical imaging assemblies can be swapped relative to the patient's eyes, thereby allowing for two different images to be captured for a single eye (allowing for capturing images of the anterior and posterior segments of a given eye).

FIG. 6 illustrates the performance characteristics and optical layout of the fundus camera of the wearable headset.

FIG. 7 illustrates the floating optical group used to correct accommodation errors and establish best focus.

FIG. 8 shows a graph that illustrates how pupil diameter varies with screen brightness.

FIG. 9 shows an exemplary embodiment of the fundus camera flexure.

FIG. 10 shows various prescription attributes of the slit lamp microscope of the wearable headset.

FIG. 11 shows the shape of slit illumination at either end of the design volume.

FIG. 12 is a block diagram illustrating an eye assessment system, including a machine learning system, consistent with the present disclosure.

FIG. 13 is a block diagram illustrating inputting of reference data (i.e., training data sets) into the machine learning system.

FIG. 14 shows a machine learning system according to certain embodiments of the present disclosure.

FIG. 15 is a block diagram illustrating receipt of one or more eye images acquired via the wearable headset, subsequent processing of the eye images via a machine learning system and image analysis module of the present disclosure, and outputting of eye health assessment to be provided to the patient.

### DETAILED DESCRIPTION

By way of overview, the present invention is directed to a system for providing a remote ophthalmologic examination and assessment of a patient's eyes. More specifically, aspects of the invention may be accomplished using a portable, wearable headset allowing a person to perform self-administered collection of eye image data for use in a remote ophthalmologic examination and assessment of the person's eye health.

In particular, the wearable headset of the present invention is a small, portable, and low-cost eye-imaging device that combines the functions of multiple imaging devices, without sacrificing quality. The headset is capable of capturing images of both the anterior and posterior segments of a person's eye without requiring involvement of a trained operator or technician. This portability and self-imaging capability allows for a person to capture digital images of their eyes without having to travel to a clinical setting and obtain assistance. Rather, a person can capture images from the comfort of their home in a relatively automated fashion. The invention further allows for the digital images to be provided to a computing system operably associated with the headset and which provides an interactive platform with which a medical professional is able to interact and analyze the one or more digital images for diagnosis and monitoring of a condition status of the at least one of the patient's eyes.

Accordingly, the system of the present invention, including the wearable headset, enables patients to undergo a complete and fully automated eye examination, while in a fully remote manner. The portability and self-imaging capacity of the wearable headset will allow providers to conduct home-based examinations, thereby removing barriers associated with the current paradigm of in-clinic eye care. This technology allows providers to screen and monitor for potentially blinding conditions such as age-related macular degeneration, diabetic retinopathy, and glaucoma, all in a remote manner. In some embodiments, the system may further be configured to provide automated analysis of the digital images and diagnose a condition status of an eye, including early diagnosis and treatment of an eye disease, based on artificial intelligence techniques.

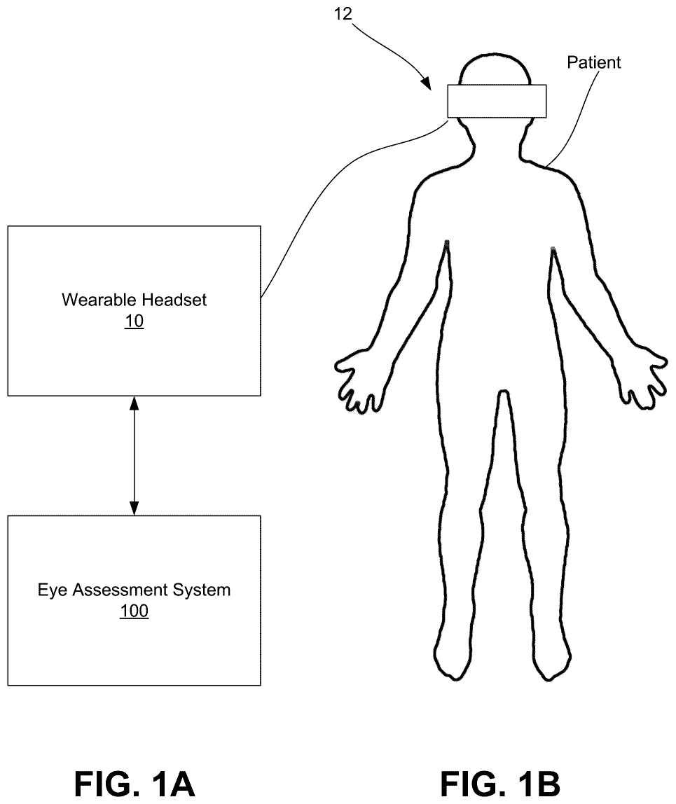

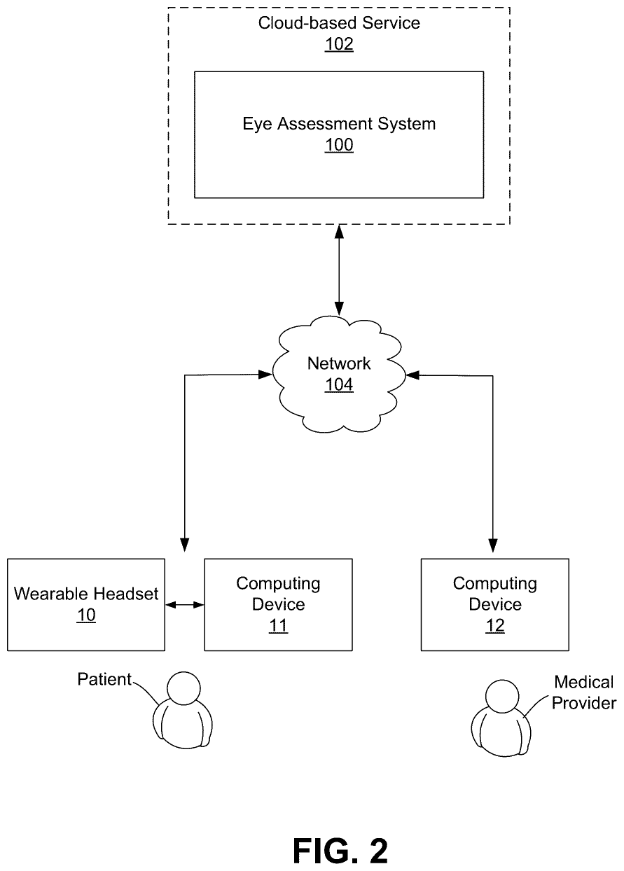

FIGS. 1A and 1B are diagrammatic illustrations of a system for providing a remote ophthalmologic examination and assessment of a patient's eyes. FIG. 2 is a block diagram illustrating the system of the present invention in more detail. As shown, the system includes a portable, wearable headset 10 allowing a person to perform self-administered collection of eye image data and an eye assessment system 100 operably associated with the headset and which allows for analysis and subsequent assessment of a health of the patient's eyes.

As previously described, a person may utilize the wearable headset 10 to capture digital images of both the anterior and posterior segments of both eyes without requiring involvement of a trained operator or technician. The wearable headset is able to communicate (either via wired or wireless communication means) with a computing device 11 and provide digital image thereto. The computing device 11 may be integrated within the headset itself or may be a separate component (e.g., a PC, laptop, tablet, smartphone or the like). In turn, the invention further allows for the digital images to be provided to the eye assessment system 100 use in an ophthalmologic examination and assessment of the person's eyes based on analysis of the digital images. For example, as shown, the eye assessment system 100 may be embodied on a cloud-based service 102, for example. The eye assessment system 100 is configured to communicate and share data the wearable headset 10. It should be noted, however, that the system 100 may also be configured to communicate and share data with the computing device 11 associated with the patient.

In some embodiments, the eye assessment system 100 may provide an interactive platform with which a medical professional is able to interact and analyze the one or more digital images for diagnosis and monitoring of a condition status of the at least one of the patient's eyes. For example, as shown in FIG. 2, the system 100 may be configured to communicate with a medical provider via a computing device 12 associated with the medical provider. The computing device 12 may include a PC, laptop, tablet, smartphone or the like. In the present context, the medical provider may include a clinician, such as a physician, physician's assistant, nurse, or other medical professional trained to provide ophthalmologic examinations and assessments. The system 100 is configured to communicate and exchange data with the wearable headset 10 and computing devices 11 and 12 over a network 104, for example.

The network 104 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the wearable headset 10 and computing device 11 and/or between the wearable headset 10, computing device 11, system 100, and computing device 12 may be, in whole or in part, a wired connection.

The network 104 may be any network that carries data. Non-limiting examples of suitable networks that may be used as network 18 include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G), fifth generation (5G), and future generations of cellular-based data communication technologies, Bluetooth radio, Near Field Communication (NFC), the most recently published versions of IEEE 802.11 transmission protocol standards, other networks capable of carrying data, and combinations thereof. In some embodiments, network 104 is chosen from the internet, at least one wireless network, at least one cellular telephone network, and combinations thereof. As such, the network 104 may include any number of additional devices, such as additional computers, routers, and switches, to facilitate communications. In some embodiments, the network 104 may be or include a single network, and in other embodiments the network 104 may be or include a collection of networks.

It should be noted that, in some embodiments, the system 100 is embedded directly into a remote server or computing device, or may be directly connected thereto in a local configuration, as opposed to providing a web-based application. For example, in some embodiments, the system 100 operates in communication with a medical setting, such as an examination or procedure room, laboratory, or the like, may be configured to communicate directly with the wearable headset 10, and thereby control operation thereof either via a wired or wireless connection.

As will be described in greater detail herein, the wearable headset is a patient wearable instrument that is used in remote assessment of eye health. Functions that would be provided by a slit lamp and/or fundus camera in a clinical setting are provided by a lightweight, head mounted device that can be deployed in a variety of home settings and industrial environments. Accordingly, a remotely located ophthalmologist (or other medical provider associated with an eye examination and assessment) can perform real time diagnostic procedures using already familiar controls and imagery associated with slit lamps, ophthalmoscopes, and fundus cameras. The wearable headset is configured to deliver consistent imagery with improved resolution, contrast, and illumination than conventional instruments.

It should also be noted that capturing of digital images may occur offline. In other words, a patient may use the wearable headset to capture digital images of their eyes in an offline mode (i.e., without a medical provider concurrently analyzing the digital images in real, or near real, time. Accordingly, digital images can be saved and reviewed at a later point in time. Furthermore, digital images may further undergo post processing enhancement or the like. Consistent imagery also facilitates development of standard image processing pipelines and even development of training sets for machine learning, as discussed in greater detail herein.

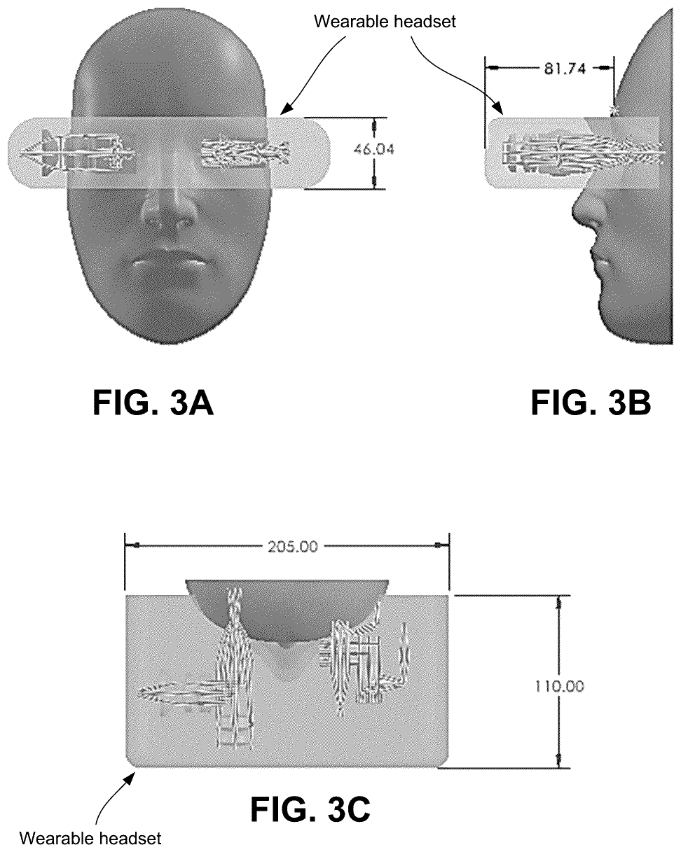

Exemplary embodiments of a wearable headset consistent with the present disclosure are illustrated in FIGS. 3A-3C, 4A-4C, and 5.

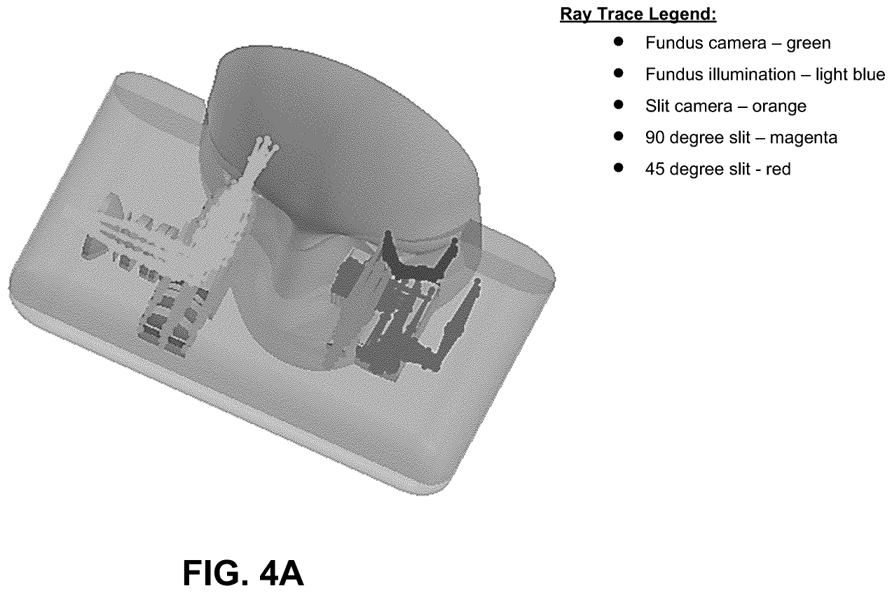

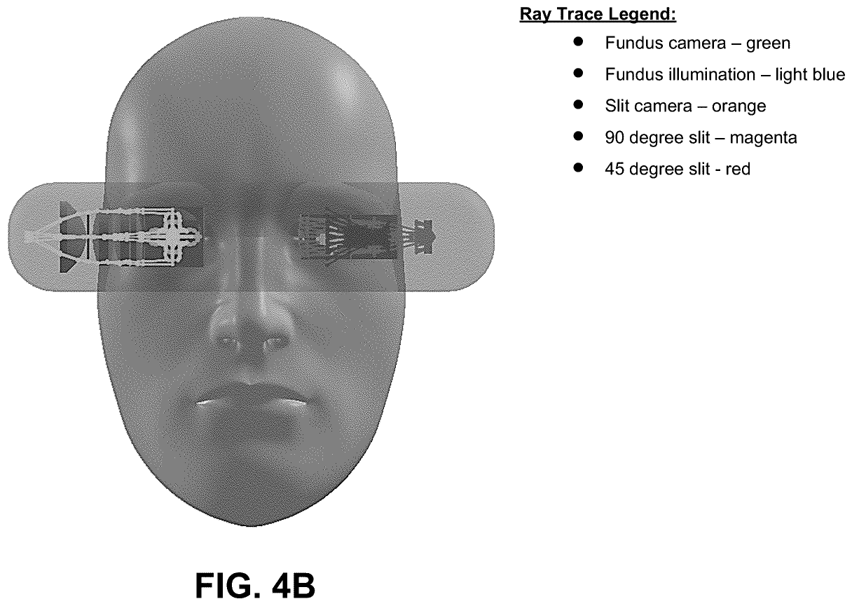

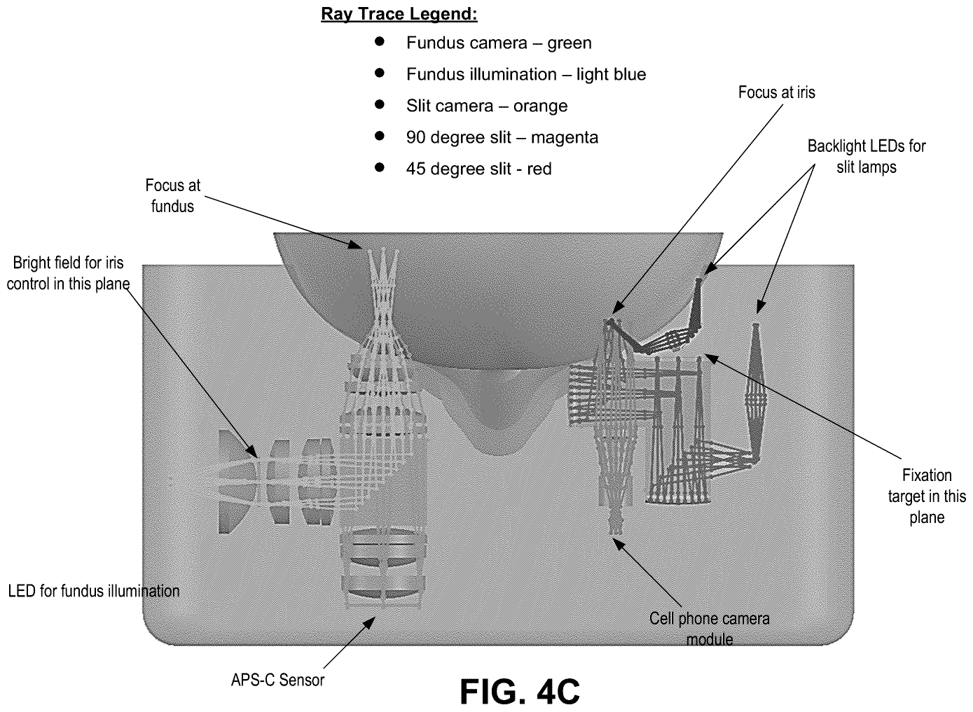

For example, FIGS. 3A, 3B, and 3C show front facing, side, and plan views of a wearable headset consistent with the present disclosure. As shown, the wearable headset is sized to fit upon a person's face and thereby position first and second optical imaging assemblies over the respective eyes. FIGS. 4A, 4B, and 4C are perspective, front facing, and plan views of the wearable headset illustrating the various components of the first and second optical imaging assemblies.

As shown, the portable, wearable headset includes a first optical imaging assembly for capturing one or more images of an anterior segment of at least one of the person's eyes and a second optical imaging assembly for capturing one or more images of a posterior segment of at least one of the person's eyes.

For example, the first optical imaging assembly may generally be configured to capture image data providing visualization of one or more structures within the anterior segment of an eye, including, but not limited to, at least one of a cornea, iris, ciliary body, and lens. In one embodiment, the first optical imaging assembly may include a slit lamp module. The second optical imaging assembly may generally be configured to capture image data providing visualization of one or more structures within the posterior segment of an eye, including, but not limited to, vitreous humor, retina, choroid, and optic nerve. In one embodiment, the second optical imaging assembly may include a fundus camera module.

As shown, the optical imaging assemblies are monocular in nature and evaluations are performed one eye at a time. For example, the first optical imaging assembly (e.g., the slit lamp module), may be dedicated to evaluation of the cornea, crystalline lens, and other anterior structures. The second optical imaging assembly (e.g., the fundus camera module), may be dedicated to evaluation of the macula, fovea, arcades, and other posterior structures.

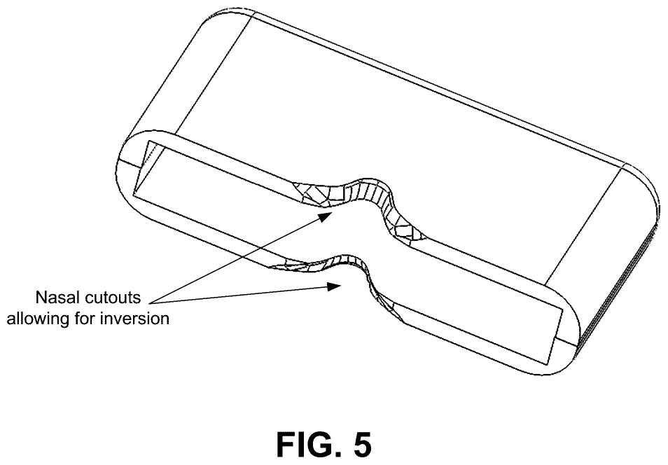

These modules may be swapped by inverting the headset. For example, the ergonomics of the headset are vertically symmetrical with respect to the patient's face. As shown in FIG. 5, for example, the headset comprises a frame supporting the first and second optical imaging assemblies relative to the person's eyes. When in a first orientation, the first and second optical imaging assemblies are positioned relative to the person's right and left eyes, respectively. When in a second orientation, the first and second optical imaging assemblies are positioned relative to the left and right eyes, respectively.

To allow this invertible functionality, the frame of the headset comprises an invertible nose bridge provided between the first and second optical imaging assemblies. Accordingly, the headset can be worn in the first and second orientations by rotating the headset 180 degrees in a plane of the first and second optical imaging assemblies. For example, invertible nose bridge comprises a first recess and an opposing second recess (shown as nasal cutouts), each of the first and second recesses being shaped and/or sized to receive a portion of the person's nose and are symmetrical relative to one another. When in the first orientation, the first recess is positioned adjacent to an upper portion of the person's nose and the second recess is positioned adjacent to a lower portion of the person's nose. When in the second orientation, the second recess is positioned adjacent to the upper portion of the person's nose and the first recess is positioned adjacent to the lower portion of the person's nose.

Accordingly, when in the first orientation, the first optical imaging assembly is configured to capture image data providing visualization of one or more structures within the anterior segment of the right eye and the second optical imaging assembly is configured to capture image data providing visualization of one or more structures within the posterior segment of the left eye. When in the second orientation, the first optical imaging assembly is configured to capture image data providing visualization of one or more structures within the posterior segment of the left eye and the second optical imaging assembly is configured to capture image data providing visualization of one or more structures within the anterior segment of the right eye.

Accordingly, the invertible nose bridge of the frame of the headset allows for the headset to be inverted such that, upon rotating the headset 180 degrees, the first and second optical imaging assemblies can be swapped relative to the patient's eyes, thereby allowing for two different images to be captured for a single eye (allowing for capturing images of the anterior and posterior segments of a given eye).

As such, during an imaging procedure, the patient is required to remove, invert, and replace the headset mid exam. This process swaps the fundus camera module to the eye formerly examined with the slit lamp module and vice versa.

As shown in FIGS. 4A, 4B, and 4C, each of the optical imaging assemblies (i.e., the slit lamp module and fundus camera module) has optical subsystems that are identified and color coded (refer to ray trace legend). The modules are generally configured to operate independently with one exception: fixation targets may be presented to the “other eye” or “fellow eye” in some procedures. In such an event, illuminators from both modules could be in operation simultaneously. Referring to FIG. 4C, specific components of the first and second optical imaging assemblies are shown and coincident ray traces are provided.

The fundus camera module comprises a fundus illumination assembly including a light source and at least one optical element for projecting illumination upon the posterior segment of a respective eye. The fundus camera module further comprises an imaging assembly for capturing one or more images of the posterior segment illuminated via the fundus illumination assembly.

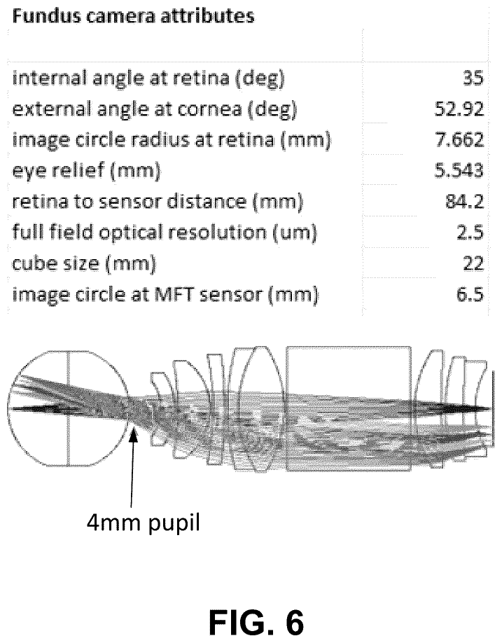

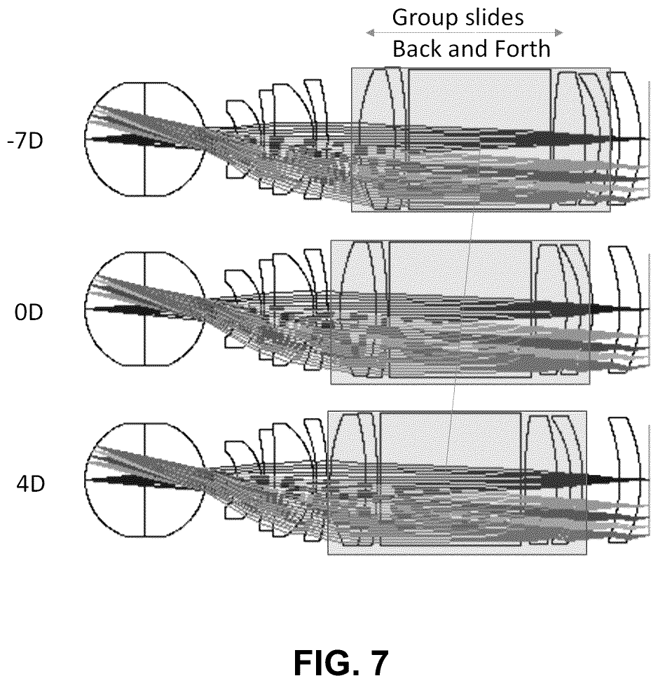

FIG. 6 illustrates the performance characteristics and optical layout of the fundus camera of the wearable headset. FIG. 7 illustrates the floating optical group used to correct accommodation errors and establish best focus. The floating optical group, inside the blue box, translates a short distance to span an extremely large −7 D to +4 D focus range. This is very convenient for motorized actuation that will be required for remote operation. It is also very space efficient. Such floating groups are novel to fundus cameras.

The fundus illuminator is jointly designed with the fundus camera. Illumination is folded into the camera's imaging path using a polarizing beam splitter. At the patient's eye, the illumination path and imaging path are co-axial and have orthogonal, or crossed, polarization. Crossed polarization extinguishes specular reflections from the cornea, allowing higher contrast imaging of the fundus. An illumination scheme typical of projectors is used for evenly lighting the fundus, it is known as Kohler illumination. Kohler illumination reimages the LED light source into the iris. Magnification at the iris is chosen so that all light can pass through the undilated iris. In addition to uniform illumination, Kohler illumination ensures illumination light does not backreflect off the iris and compete with fundus imagery.

When the fundus illuminator is in operation the patient sees the image of large (35 degree FOV) white screen. This image corresponds to a reticle or slide labelled “Bright field” in FIG. 4C. Linework on this reticle could present a fixation target to the user.

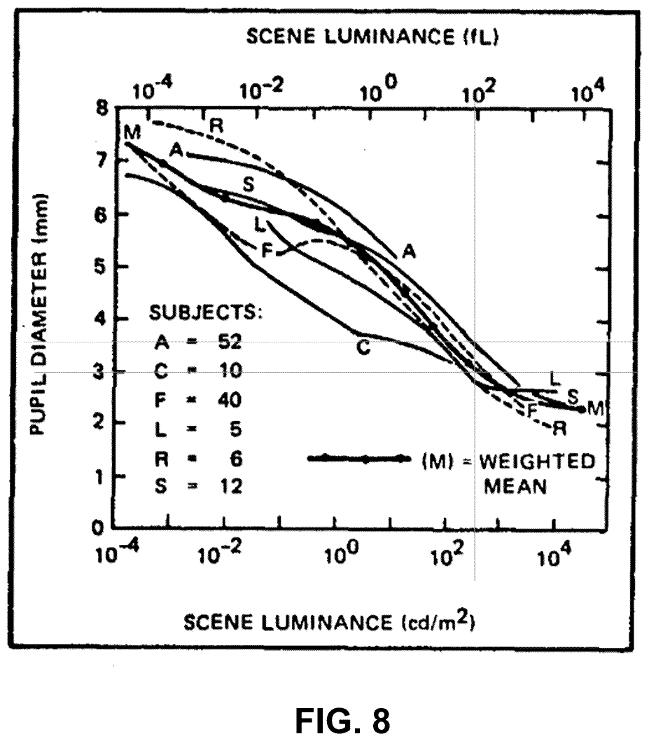

The more important function of the white screen is the ability to actively control the iris diameter. FIG. 8 shows a graph that illustrates how pupil diameter varies with screen brightness. For brightness greater than 100 cd/m\hat{ }2, pupil diameter is very consistent across a number of studies. By adjusting LED brightness in the fundus illuminator, the pupil can be “set” to a desired diameter. When the pupil diameter is set to the nominal pupil size of the fundus camera prescription (4 mm in this example), optimal sharpness is achieved. The use of a deterministic physiologic response is novel and has not been done before. Control of the iris diameter is also vital to undilated operation. If the pupil is too small it will be overfilled by the Kohler illumination. In such a case stray light will affect fundus camera contrast and possibly introduce other artifacts.

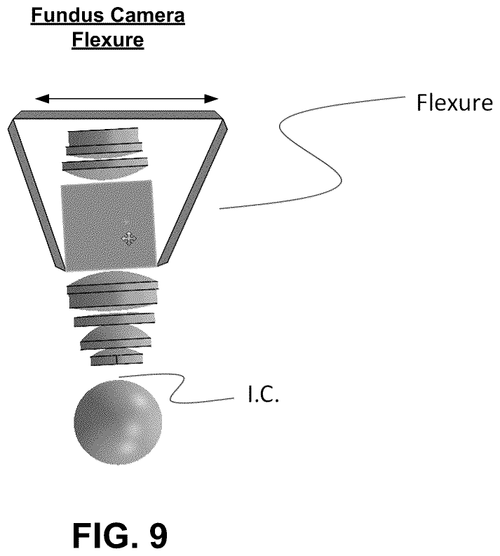

The fundus camera is compatible with a miniature flexure that rotates the fundus camera and its illuminator about an instantaneous center at the patient's iris. FIG. 9 shows an exemplary embodiment of the fundus camera flexure. While not a pure rotation, the locus of the instantaneous center is small enough to allow approximately +/−15 degrees of scan range. The advantages of including a fundus camera flexure include, but are not limited to, maintaining smaller optics, maintaining imaging even during nasal/temporal scans, both eyes are fixed straight ahead, the other eye fixates on a central fixation target, the fundus camera and the light source move concurrently with one another, and an instantaneous center (I.C.) is at the iris center.

Accordingly, the fundus camera module of wearable headset provides at least the following novel features: floating optical group allowing −7 D to +4 D adjustment for accommodation error; compatibility with a jointly designed co-axial illuminator; active control of iris diameter for maximum sharpness, dilation free operation, and size reduction (by elimination of a pupil relay); compatibility with a flexural scan mechanism that extends field of view; and integrated reticle plane for fixation targets.

As will be described in greater detail herein, the slit lamp module comprises at least a 90-degree slit lamp assembly and a 45-degree slit lamp assembly. The slit lamp module further comprises an imaging assembly for capturing one or more images of the anterior segment of a respective eye illuminated via the 90-degree and 45-degree slit lamp assemblies.

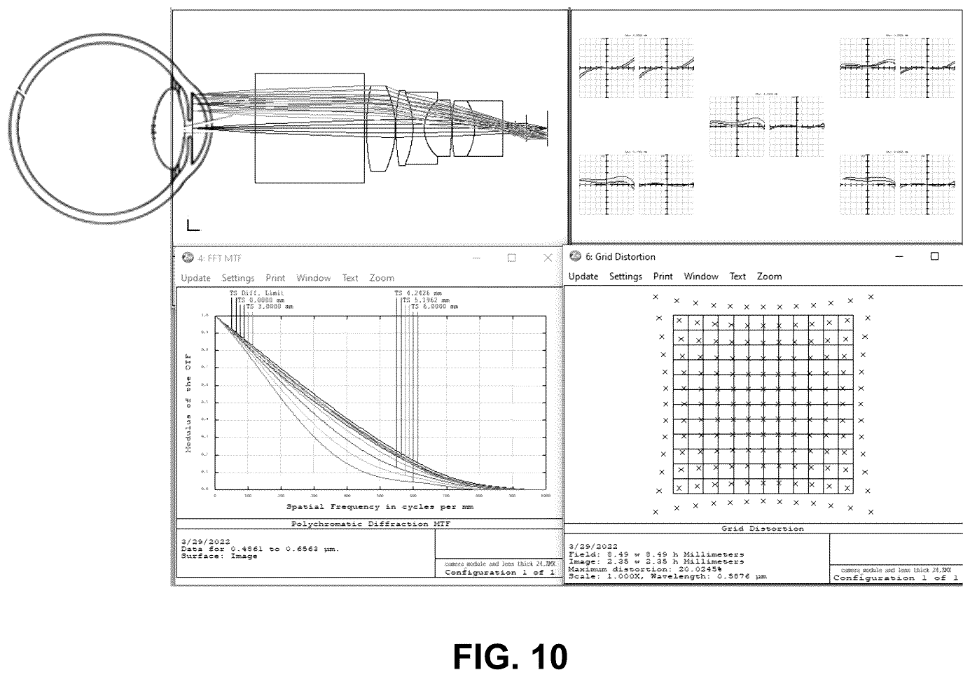

FIG. 10 shows various prescription attributes of the slit lamp microscope of the wearable headset. As shown, the slit lamp microscope features a large telecentric field of view that includes magnified images of pupil, iris, portions of the sclera, and volume of the crystalline lens including anterior and posterior surfaces. The telecentric imaging condition ensures constant magnification throughout the volume. Integrated into the slit lamp microscope is a polarizing beamsplitter used to introduce the coaxial 90-degree slit. Because slit illumination and the slit lamp microscope have crossed polarization states, specular reflections from the cornea are extinguished. When inspecting the corneal surface, an additional linear polarizer may be introduced so that specular reflections are visible.

Shapes of both 90- and 45-degree slits are formed by using a rectangular LED source commonly used in backlights. This commodity line source is much less expensive than an incandescent line lamp and runs at much lower temperatures. The 90-degree slit may be scanned by directly moving the rectangular LED source.

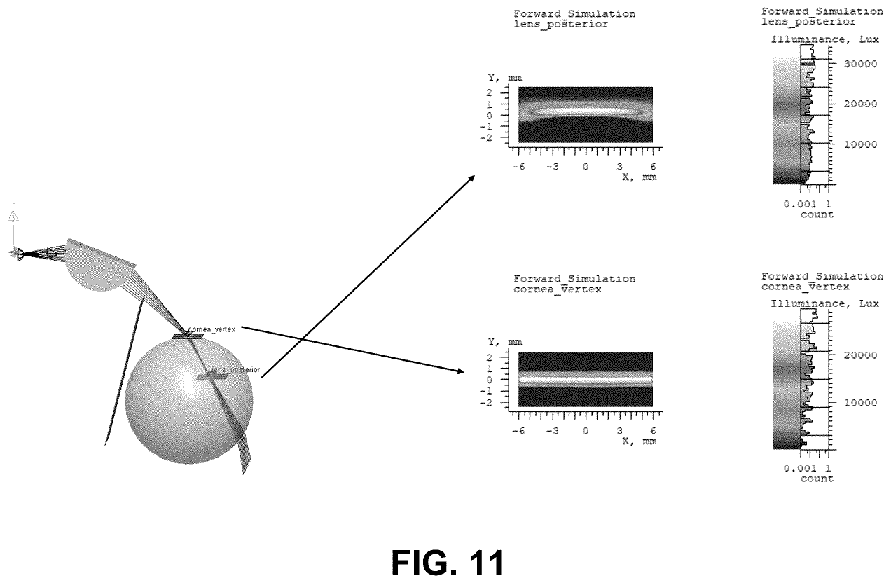

The 45 degree slit lamp uses cylindrical optics to focus the slit illumination. A novel two focal plane optimization has been performed so slit shape is well defined throughout the volume from cornea to posterior surface of crystalline lens. Shape of slit illumination at either end of the design volume is shown in FIG. 11. Scanning of 45 degree slit requires translating the small subassembly containing the LED, cylinder lens, and fold mirror.

Accordingly, the slit lamp module provides at least the following novel features: Telecentric Object Space; compatibility with a jointly designed co-axial illuminator (90-degree slit); compatibility with a jointly designed oblique illuminator (45-degree slit); and an integrated reticle plane for backlit fixation targets (90-degree slit).

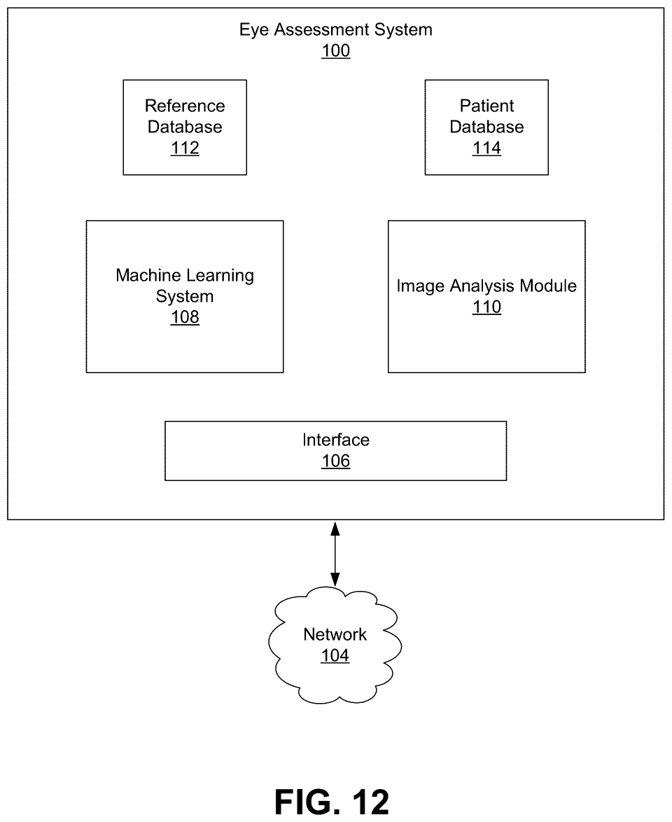

FIG. 12 is a block diagram illustrating an eye assessment system 100, including a machine learning system 108, for collecting and processing eye data, and subsequently providing eye health assessments. The system 100 is preferably implemented in a tangible computer system built for implementing the various methods described herein.

As shown, the system 100 is configured to communicate with the remote wearable headset 10 and/or the associated computing device 11 over a network 104. The system 100 is configured to receive, from the remote wearable headset 100, one or more digital images of anterior and posterior segments of at least one of the patient's eyes. The system 100 may generally be accessed by a user (i.e., the medical provider or the like) via an interface 106, for example. The interface 106 allows for a user to connect with the platform provided via the system 100 and to interact with and analyze the one or more digital images for diagnosis and monitoring of a condition status of the at least one of the patient's eyes.

The system 100 may further include one or more databases with which the machine learning system 108 communicates. In the present example, a reference database 112 includes stored reference data obtained from a plurality of training data sets and a patient database 114 includes stored sample data acquired as a result of evaluations carried out via the system 100 on a given patient's eye images. The system 100 further includes an image analysis module 110 for providing semi- or fully automated analysis and subsequently providing an eye health assessment based on analysis carried out by the machine learning system 108, as will be described in greater detail herein.

For example, in some embodiments, the system 100 allows for a medical provider to access eye image data (i.e., digital images of a patient's eyes captured via the wearable headset) and further analyze such images to make a determination of the patient's eye health (i.e., a condition of the patient's eyes). For example, via their computing device 12, the system 100 may grants a medical professional access to the one or more digital images based, at least in part, on HIPAA-compliant security measures. Upon gaining access, the interactive platform of the system 100 allows for a medical provider view images in either a live mode (i.e., view images in real time as they are being captured via the wearable headset) or in an offline mode (i.e., view images that have been previously captured at an earlier point in time).

The platform further allows for a medical provider to schedule remote, virtual meetings between the patient and medical provider. In such a scenario, the remote, virtual meeting can be synchronized with real time capturing of the one or more digital images via the remote, wearable headset. For example, the system 100 is configured to receive the one or more digital images from the wearable headset in real, or near-real, time during the remote, virtual meeting and the medical professional is able to interact with the one or more digital images via the interactive platform during the remote, virtual meeting from their computing device 12. The medical professional can then analyze the images and make an assessment of eye health without the use of the machine learning system 108.

However, in some embodiments, the system 100 is further configured to provide automated or semi-automated analysis of the one or more digital images and diagnosis of a condition status based on the analysis.

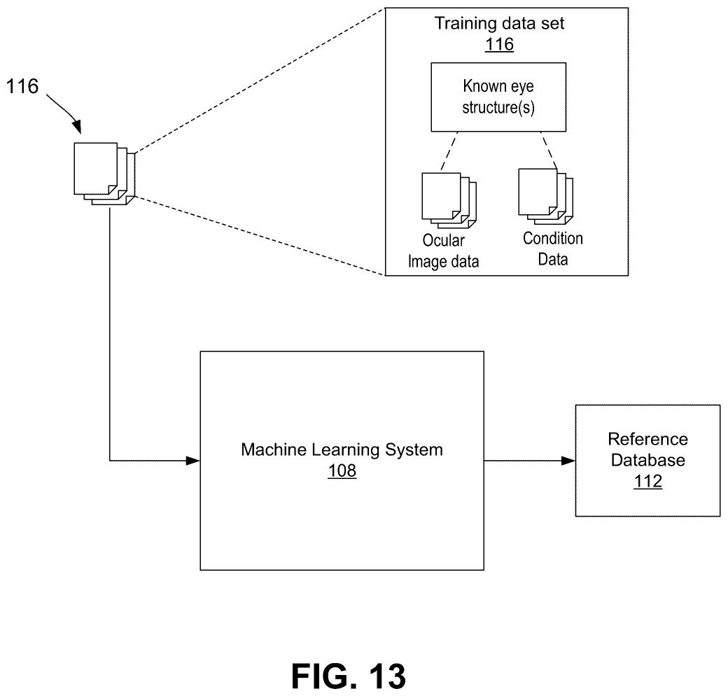

For example, the system 100 may be configured to runs a neural network that has been trained using a plurality of training data sets that include qualified reference data. FIG. 13 is a block diagram illustrating inputting of reference data (i.e., training data sets) into the machine learning system 108, for example. The machine learning techniques of the present invention, and the subsequent analysis of eye images based on such techniques, utilize reference data. The reference data may include a plurality of training data sets 116 inputted to a machine learning system 108 of the present invention. For example, each training data set includes reference eye image data, which may include, for example, eye images that include known eye structures or components. Each training data set further includes known condition data associated with the known eye structures or components. The condition data may include, for example, a condition status of a known type of eye structure or component of a given reference eye image. The condition status may include a normal condition (i.e., unremarkable or otherwise healthy condition for eye structure or component within the anterior and/or posterior segments of the eye) or an abnormal condition (i.e., an eye structure or component exhibiting certain physical characteristics associated with damage or a disease state or other undesired condition requiring medical treatment).

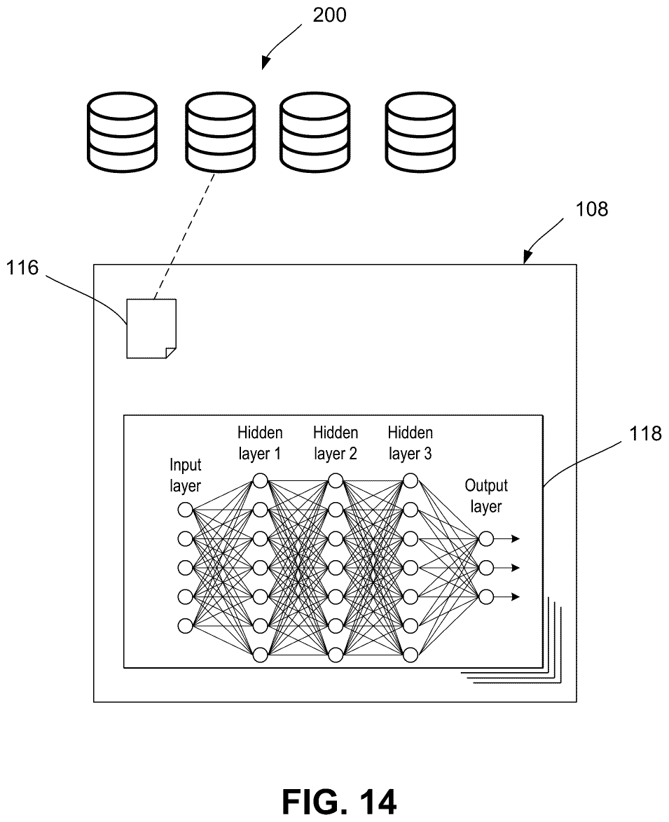

FIG. 14 shows a machine learning system 108 according to certain embodiments of the present disclosure. The machine learning system 108 accesses reference data from the one or more training data sets 116 provided by any known source 200. The source 200 may include, for example, a laboratory-specific repository of reference data collected for purposes of machine learning training. Additionally, or alternatively, the source 200 may include publicly available registries and databases and/or subscription-based data sources.

In preferred embodiments, the plurality of training data sets 116 feed into the machine learning system 108. The machine learning system 108 may include, but is not limited to, a neural network, a random forest, a support vector machine, a Bayesian classifier, a Hidden Markov model, an independent component analysis method, and a clustering method.

For example, the machine learning system 108 an autonomous machine learning system that associates the condition data with the reference eye image data. For example, the machine learning system may include a deep learning neural network that includes an input layer, a plurality of hidden layers, and an output layer. The autonomous machine learning system may represent the training data set using a plurality of features, wherein each feature comprises a feature vector. For example, the autonomous machine learning system may include a convolutional neural network (CNN). In the depicted embodiment, the machine learning system 108 includes a neural network 118.

The machine learning system 108 discovers associations in data from the training data sets. In particular, the machine learning system 108 processes and associates the reference image data and condition data with one another, thereby establishing reference data in which image characteristics of known eye structures or components are associated with known conditions of the eye structures or components. The reference data is stored within the reference database 112, for example, and available during subsequent processing of a patients eye images received from the wearable headset.

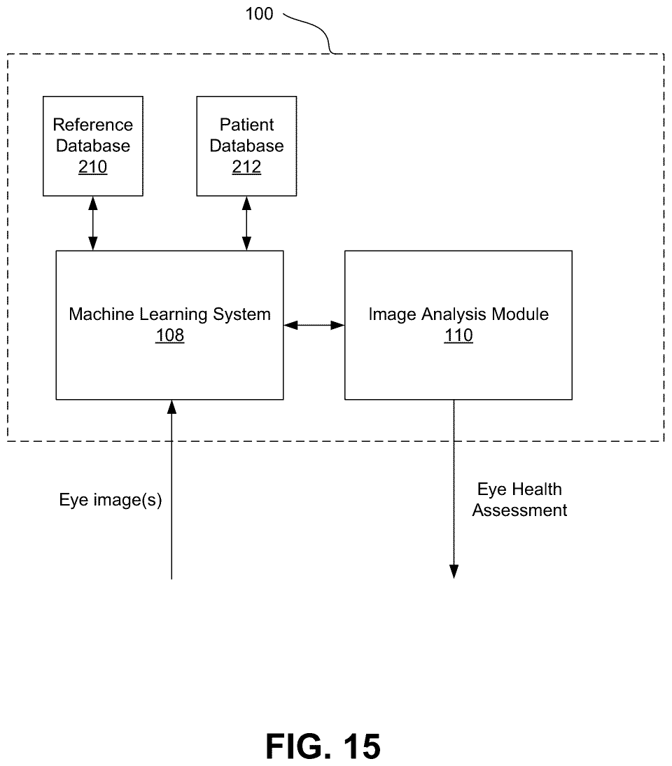

FIG. 15 is a block diagram illustrating receipt of one or more eye images acquired via the wearable headset, subsequent processing of the eye images via a machine learning system 108 and image analysis module 110 of the present disclosure, and outputting of eye health assessment to be provided to the patient.

As shown, the system 100 is configured to receive images of one or both of the patient's eyes having undergone self-administered collection of eye images via the wearable headset. Upon receiving the eye images, the system 100 is configured to analyze the images using the neural network of the machine learning system 108 and based on an association of the condition data with the reference eye image data. Based on such analysis, the computing system is able to identify one or more eye structures within the eye image (within both anterior and posterior segments of a given eye) and further identify a condition associated with eye structures identified. More specifically, the machine learning system 108 correlates the patient's eye image data with the reference data (i.e., the reference image data and condition data). For example, the machine learning system 108 may include custom, proprietary, known and/or after-developed statistical analysis code (or instruction sets), hardware, and/or firmware that are generally well-defined and operable to receive two or more sets of data and identify, at least to a certain extent, a level of correlation and thereby associate the sets of data with one another based on the level of correlation.

In turn, a condition status of a patient's eyes can be determined, and a health assessment report (which provides the health assessment) can be provided to the patient and/or the medical provider via associated computing devices. The condition status of a patient's eyes may be noted as a normal condition or an abnormal condition. For example, an abnormal condition may include, or is otherwise associated with, a disease. The disease may be associated with the eye, such as age-related macular degeneration or glaucoma. In some instances, the disease may include diabetes mellitus. In particular, the condition may include diabetic retinopathy.

Accordingly, the system of the present invention, including the wearable headset, enables patients to undergo a complete and fully automated eye examination, while in a fully remote manner. The portability and self-imaging capacity of the wearable headset will allow providers to conduct home-based examinations, thereby removing barriers associated with the current paradigm of in-clinic eye care. This technology allows providers to screen and monitor for potentially blinding conditions such as age-related macular degeneration, diabetic retinopathy, and glaucoma, all in a remote manner. In some embodiments, the system may further be configured to provide automated analysis of the digital images and diagnose a condition status of an eye, including early diagnosis and treatment of an eye disease, based on artificial intelligence techniques.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A portable, wearable headset for use in providing a remote and self-administered collection of data for use in an ophthalmologic examination and assessment of one or more eyes of a person wearing the headset, the headset comprising:
a first optical imaging assembly for capturing one or more images of an anterior segment of at least one of the person's eyes; and
a second optical imaging assembly for capturing one or more images of a posterior segment of at least one of the person's eyes.

2. The wearable headset of claim 1, wherein the first optical imaging assembly is configured to capture image data providing visualization of one or more structures within the anterior segment of an eye.

3. The wearable headset of claim 2, wherein the one or more structures within the anterior segment comprise at least one of a cornea, iris, ciliary body, and lens.

4. The wearable headset of claim 1, wherein the second optical imaging assembly is configured to capture image data providing visualization of one or more structures within the posterior segment of an eye.

5. The wearable headset of claim 4, wherein the one or more structures within the posterior segment comprise at least one of vitreous humor, retina, choroid, and optic nerve.

6. The wearable headset of claim 1, wherein the headset comprises a frame supporting the first and second optical imaging assemblies relative to the person's eyes.

7. The wearable headset of claim 6, wherein:
in a first orientation, the first and second optical imaging assemblies are positioned relative to the person's right and left eyes, respectively; and
in a second orientation, the first and second optical imaging assemblies are positioned relative to the left and right eyes, respectively.

8. The wearable headset of claim 7, wherein the frame comprises an invertible nose bridge provided between the first and second optical imaging assemblies.

9. The wearable headset of claim 8, wherein the headset can be worn in the first and second orientations by rotating the headset 180 degrees in a plane of the first and second optical imaging assemblies.

10. The wearable headset of claim 8, wherein the invertible nose bridge comprises a first recess and an opposing second recess, each of the first and second recesses being shaped and/or sized to receive a portion of the person's nose and are symmetrical relative to one another.

11. The wearable headset of claim 10, wherein:
when in the first orientation, the first recess is positioned adjacent to an upper portion of the person's nose and the second recess is positioned adjacent to a lower portion of the person's nose; and
when in the second orientation, the second recess is positioned adjacent to the upper portion of the person's nose and the first recess is positioned adjacent to the lower portion of the person's nose.

12. The wearable headset of claim 7, wherein:
when in the first orientation, the first optical imaging assembly is configured to capture image data providing visualization of one or more structures within the anterior segment of the right eye and the second optical imaging assembly is configured to capture image data providing visualization of one or more structures within the posterior segment of the left eye; and
when in the second orientation, the first optical imaging assembly is configured to capture image data providing visualization of one or more structures within the posterior segment of the left eye and the second optical imaging assembly is configured to capture image data providing visualization of one or more structures within the anterior segment of the right eye.

13. The wearable headset of claim 1, wherein the first optical imaging assembly comprises a slit lamp module and the second optical imaging assembly comprises a fundus camera module.

14. The wearable headset of claim 13, wherein the slit lamp module comprises at least a 90-degree slit lamp assembly and a 45-degree slit lamp assembly.

15. The wearable headset of claim 13, wherein the slit lamp module further comprises an imaging assembly for capturing one or more images of the anterior segment of a respective eye illuminated via the 90-degree and 45-degree slit lamp assemblies.

16. The wearable headset of claim 13, wherein the fundus camera module comprises a fundus illumination assembly including a light source and at least one optical element for projecting illumination upon the posterior segment of a respective eye.

17. The wearable headset of claim 16, wherein the fundus camera module further comprises an imaging assembly for capturing one or more images of the posterior segment illuminated via the fundus illumination assembly.

18. The wearable headset of claim 1, further comprising a communication module for permitting the exchange of data between a computing device and the first and second optical imaging assemblies.

19. The wearable headset of claim 18, wherein the communication module is configured to permit wired and/or wireless transmission of data between the computing device and the first and second optical imaging assemblies.

20. The wearable headset of claim 18, wherein the computing device is a remote server configured to receive the one or more images captured via the first and second optical imaging assemblies for use in an ophthalmologic examination of the person's eyes.
